(19) United States
(12) Patent Application Publication
(10) Pub. No.: US 2011/0288060 A1
(43) Pub. Date: Nov. 24, 2011

(54) APPARATUS AND PROCESS FOR PRODUCING CRYSTALS

(75) Inventors: Graham Ruecroft, Oxfordshire (GB); John Burns, Oxfordshire (GB)

(73) Assignee: PROSONIX LIMITED, Oxford, Oxfordshire (GB)

(21) Appl. No.: 13/143,257

(22) PCT Filed: Jan. 5, 2010
(86) PCT No.: PCT/GB2010/050007

§ 371 Date: Aug. 15, 2011

(30) Foreign Application Priority Data
Jan. 6, 2009 (GB) 0900080.3

(51) Int. Cl. A61K 31/58; A61K 31/19; A61K 31/135; A61P 37/08; A61P 11/08; A61P 29/00; A61P 31/12; A61P 31/00; A61P 35/00; A61P 25/00; A61P 9/00; A61P 25/16; A61P 31/18; A61P 25/08; A61P 1/00; A61P 21/00; A61P 3/00; A61P 13/00; A61P 19/00; B01J 8/16; B01J 19/10; A61K 31/56

(52) U.S. Cl. 514/174; 514/179; 514/557; 514/653; 422/128; 204/157.62; 428/402

(57) ABSTRACT

This invention provides an oscillating baffled reactor apparatus for preparing crystalline particles of at least one substance comprising: a reactor vessel; means for supplying a first flowing stream; means for oscillating fluid within the reactor vessel; a plurality of baffles; source of ultrasonic radiation; and means for collecting said particles.

### FIELD OF INVENTION

This invention relates to an apparatus and process for producing crystals. The present invention has application in the manufacture of chemicals, such as active ingredient compounds and excipients for use in pharmaceutical formulations, such as inhalation formulations, and in the manufacture of agrochemical formulations, such as liquid-based suspensions.

### BACKGROUND OF INVENTION

The control of crystal and precipitate particle size is very important in some circumstances, in particular in the pharmaceutical and agrochemical industries in which the final product form of the active principal of interest is in the form of a fine powder. The manner in which an active principal behaves in a biological system depends upon many factors, inter alia, the size of the particle and the crystal form. Small particles may be made by processes such as milling, but such processes may have a detrimental effect on the material properties and may also produce a significant proportion of particles which are unsuitable for the desired use, for example, they may be of an inappropriate shape. Such particles may undergo morphological alterations, leading to undesirable surface polymorphological transformation which in turn may give rise to the formation of amorphous structures. The particles may become highly charged which may contribute to undermining flow-rates. Also, particles destined for use in aerosols may be comprised should they become highly charged. Crystallisation of materials in the desired size range directly from solution would be desirable. For example the small particles destined for inhaled delivery are less than 5 μm in size whereas dry powder material for tablets or capsules for oral delivery may be around 5-200 μm prior to manufacture. In all cases it is preferable to avoid mechanical milling and prepare particles of the appropriate size directly from the process solutions.

The mixing of heterogeneous phase mixtures in which a liquid phase is in contact with a further liquid, solid or gaseous phase is known in the art. WO99/55457 describes an oscillatory baffled reactor (OBR) comprising a reactor vessel, supply means to supply a substantially continuous feed of an aqueous medium through the reactor vessel; and oscillation means to oscillate liquid within the reactor vessel; said reactor vessel comprising an inlet in communication with said supply means; an outlet adaptable for communication with said supply means; a plurality of stationary baffles; and at least one port for the introduction of process components and/or initiators. This document discloses the apparatus for phase separated synthesis of particulates.

A method and apparatus for operating temperature controlled processes in an oscillatory baffled reactor (OBR) has been described in the prior art WO 2007/060412. This apparatus provides improved process control, in particular to enable controlled temperatures to be applied to a substance in different process zones of the OBR, described by a series of tubular members arranged and operatively connected in a flow system, and each process zone has temperature regulating means.

According to this invention there is provided an apparatus for controlling a process, comprising a vessel adapted to receive and discharge fluids, and having a series of tubular members, each defining a discrete process zone, arranged and operatively connected in a flow system to form at least one continuous fluid flow path having an inlet and an outlet, wherein mixing means is provided within the flow path, and wherein each zone has temperature regulating means juxtaposed thereto for effecting temperature control therein.

Therefore, the vessel can be set such that the temperature of the contents is different in different process zones or flow paths. This can be done accurately and consistently, giving greater control over the temperature of the contents of the tubular members of the vessel. This is useful in applications such as crystallisation where the accurate control of temperature has a significant impact on the end product. The preferred mixing means comprises of a plurality of baffles.

It has been known to bring about crystallisation by mixing a solvent containing a substance to be crystallised with an anti-solvent, so that after mixing the solution is supersaturated and crystallisation occurs. WO03/061816 discloses a process for preparing crystalline particles of substance which comprises mixing a flowing solution of the substance in a liquid solvent with a flowing liquid anti-solvent for said substance, in a continuous flow cell in the presence of ultrasound and collecting the resultant crystalline particles generated, characterised in that the solution and anti-solvent are delivered into the continuous flow cell in parallel contacting streams.

### SUMMARY OF INVENTION

The invention is defined in the claims.

The first aspect of the invention comprises an oscillating baffled reactor apparatus for preparing crystalline particles of at least one substance comprising: a reactor vessel; means for supplying a first flowing stream; means for oscillating fluid within the reactor vessel; a plurality of baffles; a source of ultrasonic radiation; and means for collecting said particles.

The second aspect of the invention comprises a process for preparing crystalline particles of at least one substance comprising contacting a solution of at least one solute in a solvent in a first flowing stream with an anti-solvent in a second flowing stream; subjecting the contacted streams to oscillatory motion in an oscillating baffled reactor; applying ultrasonic radiation; and collecting the particles that are generated.

The third aspect of the invention comprises a process for preparing crystalline particles of at least one substance comprising subjecting a saturated or supersaturated flowing stream of at least one solute in solution to oscillatory motion in an oscillating baffled reactor; applying ultrasonic radiation; and collecting the particles that are generated.

The fourth aspect of the invention comprises a process for processing crystalline particles of at least one substance comprising subjecting a flowing stream comprising slurry of particles to oscillating motion in an oscillating baffled reactor; applying ultrasonic radiation and collecting the particles.

The fifth aspect of the invention comprises crystalline particles of at least one substance obtainable by the process of the second, third or fourth aspect of the invention.

### BRIEF DESCRIPTION OF THE DRAWINGS

FIG. 1 shows a longitudinal sectional view of a crystallisation apparatus incorporating two separate feed stream delivery means for the solvent and anti-solvent leading into an ultrasonic continuous flow cell configured as an Ultrasonic Oscillatory Baffled Reactor (UOBR) having an ultrasonic transducer assembly.

FIG. 2 shows a longitudinal sectional view of a crystallisation apparatus which is configured similarly to the apparatus in FIG. 1 except that a multiplicity of ultrasonic modules/flow cells are positioned at various intervals along the tubular OBR.

FIG. 3 shows a longitudinal sectional view of a crystallisation apparatus incorporating two liquid feeds contacted coaxially within a single delivery means and fed into an ultrasonic continuous flow cell configured as UOBR having an ultrasonic transducer assembly.

FIG. 4 shows a longitudinal sectional view of a crystallisation apparatus incorporating two liquid feeds contacted coaxially within a single delivery means and fed into an ultrasonic continuous flow cell configured as UOBR having a multiplicity of ultrasonic transducers placed circumferentially around a cylindrical duct.

FIG. 5 shows a longitudinal sectional view of a crystallisation apparatus incorporating a liquid feed for introduction of supersaturated liquor via a single delivery means and fed into an ultrasonic continuous flow cell, configured as UOBR having an ultrasonic transducer assembly immediately after introduction of the liquor, and with either single or a multiplicity of heat exchangers, positioned along the UOBR to control the temperature of the process liquor and thereby the level of supersaturation.

FIG. 6 shows a longitudinal sectional view of a crystallisation apparatus incorporating a liquid feed for introduction of supersaturated liquor via a single delivery means and fed into an ultrasonic continuous flow cell, configured as UOBR having an ultrasonic transducer assembly immediately after introduction of the liquor, and a multiplicity thereafter, and with a multiplicity of heat exchangers positioned along the UOBR to control the temperature of the process liquor and thereby the level of supersaturation, the heat exchangers being positioned alternately with the ultrasonic continuous flow cell so as to achieve sequentially ultrasonication then cooling.

### DETAILED DESCRIPTION

The prior art uses Oscillatory Baffled Reactors (OBRs) for carrying out chemical reactions. Further prior art describes the physical process of mixing a solution comprising a solute in a solvent and an anti-solvent in the presence of ultrasonic radiation for the formation of crystalline particles. Further prior art describes method and apparatus for operating temperature controlled processes in an oscillatory baffled reactor (OBR) to provide improved process control in temperature sensitive processes.

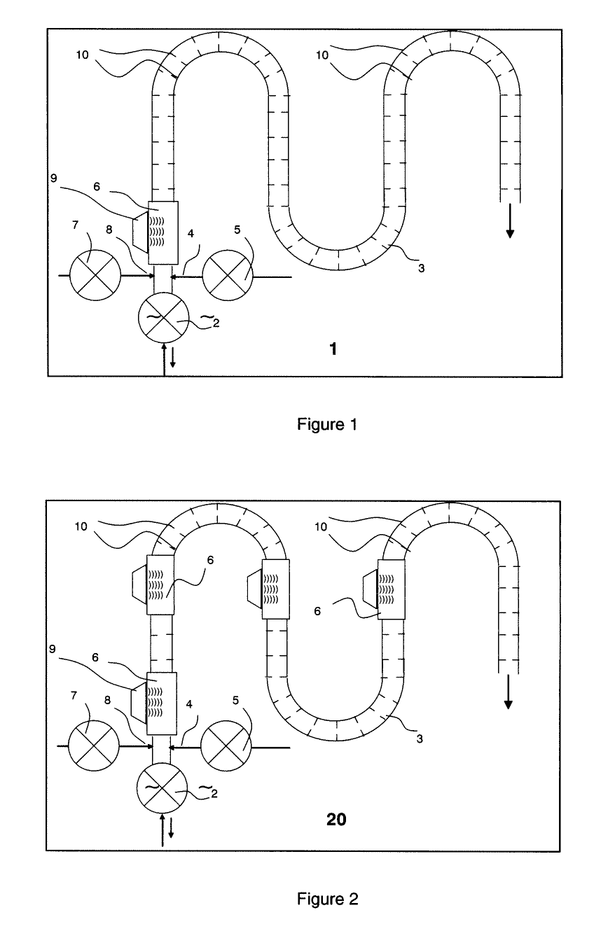

To the best of the inventors knowledge, both non-temperature regulated and temperature regulated OBRs, have not been used in the presence of ultrasound. An advantage of carrying out the crystallisation process of a substance, that is a physical process rather than a chemical process, in a UOBR is that this results in near plug flow chemistry which leads to mixing in the radial direction in the reactor vessel and no mixing in the axial direction which enables control of the resulting particle sizes produced. Further the sononucleation from the ultrasound radiation and the particle disruption from the oscillation lead to crystal nucleation at the expense of crystal growth which leads to control of the resulting particle sizes and/or particle size distributions produced. These phenomena are equally applicable to both constant temperature antisolvent and thermoregulated crystallization processes. The mixing intensity in the system can be used to influence the balance between nucleation and crystal growth and thus affect the particle size distribution produced. Control of mixing intensity is achieved through variation in the amplitude and frequency of the oscillations within the tube to provide greater specific power input to the liquids inside. The oscillatory pump, as featured in FIGS. 1 to 6, imparts motion on the constituents in an oscillatory fashion to provide this control of mixing. The main method of quantifying turbulent mixing within the system is the Reynolds number (Re), defined by the equation below, which provides a measure of the ratio of inertial and viscous forces within the systems. The pump is adapted to impart oscillatory flow-rates which gives a Reynolds number greater than 100, more preferably greater than 500, more preferably greater than 2000, to the contents of the tubular oscillatory flow reactor, and the flow of the contents or substance in the tubular vessel is turbulent flow. The contents are then transported, with the maintenance of vigorous mixing, along the length of the tubular oscillatory flow reactor. The mixture can be drawn off at a suitable outlet port.

Re=(ρ·a·f·d·)/μ

where:

| | | |
|---|---|---|
| ρ | Density | kg · m⁻³ |
| μ | Viscosity | kg · m⁻¹ · s⁻¹ |
| a | Amplitude | m |
| f | Frequency | s⁻¹ |
| d | Diameter | m |

The apparatus of this invention can be used to produce crystalline particles of a required size. The process of this invention provides a controlled way of producing crystalline particles of a required size.

Unlike the prior art, the presence of ultrasound induced cavitation assists in ensuring dispersion of the different phases, whether miscible or immiscible, solid or liquid, as well as providing powerful crystal nucleation sites this avoiding the possibility of obtaining unstable and potentially amorphous solid phases.

The second aspect of the invention comprises a process for preparing crystalline particles of at least one substance comprising contacting a solution of at least one solute in a solvent in a first flowing stream with an anti-solvent in a second flowing stream; subjecting the contacted streams to oscillatory motion; applying ultrasonic radiation; and collecting the particles that are generated.

As used herein, an anti-solvent is one in which a solid material is soluble in an amount of less than 0.1 mg/ml at 25° C., preferably less than 0.05 mg/ml at 25° C., preferably less than 0.01 mg/ml at 25° C. Conversely, as used herein, the solvent is one in which the solid material is soluble in an amount greater than 0.1 mg/ml at 25° C., preferably greater than 0.5 mg/ml at 25° C., preferably greater than 1 mg/ml at 25° C., preferably greater than 5 mg/ml at 25° C., preferably greater than 10 mg/ml at 25° C.

The temperatures of the anti-solvent and solvent may be between −10° C. and +120° C.

The solvent and the anti-solvent may be miscible with each other, such as water and 2-propanol or ethanol and water. Alternatively, the anti-solvent and solvent may be the same liquid at different temperatures. Typically, the temperatures of the liquid may lie between −10° C. and +120° C., but with a substantial temperature difference between the two. A substantial temperature difference is for example at least 20° C., preferably at least 30° C., preferably at least 50° C., such as wherein the solvent is hot water at 80° C. and the anti-solvent is cold water at 10° C.

The solvent and anti-solvent can also be immiscible liquids.

For any given solid material, the skilled person is capable of determining suitable solvents thereof, without burden. Some examples of solvents suitable for certain solid materials are as follows. Volatile organic solvents such as methanol, ethanol, dichloromethane, ethyl acetate, acetone, 2-propanol and non-organic solvents such was water would be typical solvents for pharmaceutically active ingredients. Preferred excipients may include, for example, lactose and stearic acid. Lactose may be dissolved in water or ethanol-water mixture. Stearic acid may be dissolved in ethyl acetate or ethanol.

The anti-solvent may comprise among others, water, cyclohexane, 2-propanol, isooctane, heptane, or mixtures thereof.

Whilst not an exhaustive list, some examples of solvent and anti-solvent combinations are shown in Table 1.

TABLE 1

| Solvent (also | Anti-Solvent (also |
|---|---|
| possible anti-solvent) | possible solvent) |
| Methanol | Water |
| Acetone | Water |
| Ethanol | Water |
| Ethanol | Cyclohexane |
| Methanol | Cyclohexane |
| Ethanol | 2-propanol |
| Methanol | 2-propanol |
| Acetone | Isooctane |
| Ethyl acetate | Heptane |
| Dichloromethane | Heptane |

In the case where the anti-solvent and solvent are not miscible with each other, there is provided a means for forming an emulsion or a dispersion wherein said solute is present in a supersaturated solution within the droplets of the emulsion or dispersion so formed. Emulsions and dispersions and their formation are known in the art. Emulsions are, by definition, droplets that are stabilised in a continuous phase, for example through the use of surfactants known in the art. Dispersions can be viewed as droplets dispersed in a continuous phase wherein the droplets are not stabilised, that is to say they do not remain as droplets but after a short time coalesce forming a two phase system with a continuous phase.

In order to stabilise dispersions, it is known to add surfactants or other stabilising agents to them, enabling the formation of stabilised droplets and thereby a stable emulsion.

The generation of the emulsion or dispersion may be partially or wholly achieved by the combined action of the shearing action of the OBR and the disruption of the ultrasonic radiation. The level of input of both of these may be used as a control factor in the size of droplets formed and maintained.

The size of droplets formed in these cases may be partially or wholly used to direct the size and morphology of the resulting particles produced by the process by means of dividing up the material that will be crystallised out into discrete spherical packets. The droplets may be of an organic or inorganic liquid and a continuous phase may be aqueous or non-aqueous depending on end purpose and design. Typically, emulsions of the present invention comprise droplets of an organic liquid comprising solute (i.e. made up of at least one active principle), and the continuous phase is an aqueous phase, together forming an aqueous dispersion or emulsion.

The emulsion may optionally contain additives such as surfactants, stabilisers and dispersants, known in the art, for assisting the formation and stabilisation of the emulsion. Additives will normally be present in an amount of 0.01 to 30 w/w %, preferably 0.1 to 20 w/w %, more preferably from 0.1 to 10 w/w %, and most preferably from 0.25 to 4 w/w %.

Such surfactants, stabilisers and dispersants will be chosen according to the nature of the emulsion, and may be non-ionic, anionic, ampholytic, zwitterionic and/or cationic depending on design. Mixtures of these surfactants, stabilisers and dispersants can also be used.

A particularly preferred nonionic surfactant is derived from a straight chain fatty alcohol containing from about 16 to about 20 carbon atoms (C₁₆-C₂₀ alcohol). These and other nonionic surfactants are well known in the art, being described in more detail in Kirk Othmer's Encyclopedia of Chemical Technology, 3rd Ed., Vol. 22, pp. 360-379, “Surfactants and Detersive Systems”, incorporated by reference herein.

Preferred non-ionic stabilisers include polysorbate (Tween 80). Preferred polymeric stabilisers include cellulosics, such as hydroxypropylycellulose and hydroxypropylmethyl cellulose, povidone (PVP K30) and pluronics (F68 F127).

Preferred anionic surfactants include salts (including, for example, sodium, potassium, ammonium, and substituted ammonium salts such as mono-, di- and triethanolamine salts) of the anionic sulfate, sulfonate, carboxylate, laurylsulphate and sarcosinate surfactants. Anionic sulfate surfactants are preferred.

Suitable amphoteric surfactants for use herein include the amine oxide surfactants and the alkyl amphocarboxylic acids.

Zwitterionic surfactants can also be incorporated into the detergent compositions hereof. These surfactants can be broadly described as derivatives of secondary and tertiary amines, derivatives of heterocyclic secondary and tertiary amines, or derivatives of quaternary ammonium, quaternary phosphonium or tertiary sulfonium compounds. Betaine and sultaine surfactants are exemplary zwitterionic surfactants for use herein.

Cationic ester surfactants which may be used in this invention are preferably water dispersible compound having surfactant properties comprising at least one ester (i.e.—COO—) linkage and at least one cationically charged group. Other suitable cationic ester surfactants, including choline ester surfactants, have for example been disclosed in U.S. Pat. Nos. 4,228,042, 4,239,660 and 4,260,529. Suitable cationic surfactants further include the quaternary ammonium surfactants selected from mono C₆-C₁₆, preferably C₆-C₁₀ N-alkyl or alkenyl ammonium surfactants wherein the remaining N positions are substituted by methyl, hydroxyethyl or hydroxypropyl groups.

The emulsion droplets typically vary in diameter from approximately 0.05 to 80 μm. Droplets with diameters in the range of 0.3 to 80 μm are known as “macro-droplets”, and the emulsions “macro-emulsions”. Droplets with diameters in the range 0.05 to 0.3 μm are known as “micro-droplets” and the emulsions as “micro-emulsions”. For the purpose of the present invention, the terms “droplet” and “emulsion” as used herein encompass both macro- and micro-droplets and macro- and micro-emulsions.

The organic liquid phase of the droplet will preferably be water insoluble. “Water insoluble” in this context means anything less than fully water miscible, preferably water immiscible, though in some situations, the organic liquid phase will dissolve in water typically in an amount of not more than 10% w/w at a temperature at which crystallisation can take place.

The emulsion may further contain a buffering agent, such as sodium acetate and acetic acid, for maintaining the pH of the emulsion at a desired level, anti-freeze agents and solubility adjusting agents, as known in the art; and may also contain a solubiliser for an active principal or principals, such as acetone or dichloromethane or a mixture of cetyl alcohol and dichloromethane, which can be easily removed following crystallisation and re-used.

Formation of the original emulsion may be carried out in the ultrasonic oscillatory baffled reactor (UOBR) in which dispersion, emulsification and crystallisation takes place. To ensure the solution (and so the emulsified droplets) becomes supersaturated, the solution prior to forming an emulsion is prepared in a saturated or near saturated state either:
(i) at a temperature which is either higher than the temperature of the anti-solvent (for example 1-20° C. higher if the solubility increases with the temperature); or lower than the temperature of the anti-solvent (for example 1-20° C. lower, if the solubility decreases with the temperature)) and thereby generating super-saturation when it is contacted with the cooler or warmer anti-solvent;
(ii) at a cooler temperature to the anti-solvent where the solvent is more soluble in the anti-solvent at the temperature of the anti-solvent thus leading to the removal of solvent from the emulsified droplet by evaporation, boiling or other means such as diffusion into the antisolvent and hence super-saturation occurs within the emulsified droplet.

(iii) at a warmer or similar temperature to the anti-solvent when the initial solution also comprises a miscible co-solvent at a temperature above the melting point of the solute, thereby generating emulsified melt droplets as the miscible co-solvent disperses into the anti-solvent.

(iv) at a similar temperature to the anti-solvent when the solvent is partially soluble in the anti-solvent and supersaturation is generated by partial extraction of solvent from the droplet. This may be applied in conjunction with various techniques, such as distillation and evaporation, that may remove solvent from the anti-solvent to allow eventual removal of all of the solvent.

(v) at a similar temperature to the anti-solvent when the anti-solvent is partially soluble in the solvent or contains a second anti-solvent miscible in both the primary anti-solvent and/or the solvent. Supersaturation is achieved by means of diffusion of the surrounding anti-solvent into the suspended droplet.

In each case as super-saturation is generated, the application of ultrasound, assisted by oscillatory motion mediates nucleation that leads to the formation of crystals.

By manipulating the flow rate ratio of anti-solvent to solvent in the process of the present invention the inventors have now made it possible to provide crystals of active principals of interest of a desired size of up to about 1000 μm (1 mm) in size.

For drugs particles destined for use as inhaled medicines the mean diameter size of particles that are able to be attained using the method of the invention lies in the range of from about 500 nm up to about 10 μm, preferably from about 600 nm to about 5 μm and most preferably from about 650 nm to about 2 μm, for example, about 700 nm or about 1 μm. Preferably the drugs particles destined for use as inhaled medicines of the invention have a volume diameter of less than 10 μm, more preferably at least 90 wt % of the active ingredient particles in a given composition have a diameter equal to or lower than 10 μm as determined by measuring the characteristic equivalent sphere diameter, known as volume diameter, by laser diffraction as described below, preferably using a Malvern or equivalent apparatus. The parameters taken into consideration are the volume diameters (VD) in microns of 10%, 50% and 90% of the particles expressed as d(10), d(50) and d(90), respectively, which correspond to the mass diameter assuming a size independent density for the particles.

Preferably no more than 10 wt % of said particles have a volume diameter d(10) lower than 0.8 μm, preferably no more than 50 wt % of said particles have a volume diameter d(50) lower than 2.0 μm, preferably at least 90 wt % of said particles have a volume diameter d(90) equal to or lower than 10 μm. Preferably 100 wt % of said particles have a volume diameter equal to or lower than 10 μm.

For drugs particles destined for use in tablets of capsules the mean diameter size of particles that are able to be attained using the method of the invention lies in the range of from about 2 μm up to about 900 μm, preferably from about 5 μm to about 400 μm and most preferably from about 10 μm to about 200 μm, for example, about 50 μm or about 100 μm. Preferably no more than 10 wt % of said particles have a volume diameter d(10) lower than 50 μm, preferably no more than 50 wt % of said particles have a volume diameter d(50) lower than 250 μm, preferably at least 90 wt % of said particles have a volume diameter d(90) equal to or lower than 900 μm. Preferably 100 wt % of said particles have a volume diameter equal to or lower than 900 μm.

The particle size can be measured by laser diffraction techniques. Light from a laser is shone into a cloud of particles, which are suspended in a transparent gas such as air. The particles scatter the light; smaller particles scattering the light at larger angles than bigger particles. The scattered light can be measured by a series of photodetectors placed at different angles. This is known as the diffraction pattern for the sample. The diffraction pattern can be used to measure the size of the particles using well documented light scattering theory. The particles are assumed to be spherical but few particles are actually spherical. The particle diameters are calculated from the measured volume of the particle, but assume a sphere of equivalent volume.

The flow rate ratio of anti-solvent:solvent (the “flow rate ratio” hereinafter) of the invention is preferably higher than 1:1, and may be of any flow rate ratio depending on design and the end purpose for the crystals that are obtained using the process of the invention. The flow rate ratio employed in the process of the invention may be decided taking into account the substance of interest, the desired size of the crystals required for a given purpose, and how the crystals are to be administered to a subject, such as to a mammal (e.g. a human being, an equine, bovine or ovine animal) in the form of a suitable medicament, or to a plant in the form of a suitable agrochemical, for example a pesticide, a herbicide, a fungicide, bactericide, or a virucide.

Suitable flow rate ratios for use in the process of the invention may be any flow rate ratio of the anti-solvent stream:solvent stream, up to 1000:1, for example, 900:1, 800:1, 700:1, 600:1, 500:1, 400:1, 300:1, 200:1, 100:1, 50:1, 40:1, 30:1, 10:1, 5:1 or 1:1 and the like or any flow rate ratio there between, such as 380:1, 330:1, 333:1, 165:1, 80:1, 25:1, 3:1 and the like. The flow rate ratio will be governed by the size of the crystals that are required for a given end purpose and the proposed delivery vehicle for them that is to be used in a subject organism. Preferably the flow rate ratio is between 20:1 and 1000:1, more preferably 20:1 to 900:1, even more preferably 20:1 to 500:1, most preferably 20:1 to 400:1.

Typically the flow rates of the two liquid phases will govern the mixture composition which in turn will affect solubility of the solute of interest. In such a case of reasonable solubility of the solute Ostwald ripening to some degree may lead to particle growth. Reasonable solubility refers to the solubility of the solute in the steady state solvent/anti-solvent composition. In this case reasonable solubility would be greater than 1 mg/ml, preferably greater than 5 mg/ml, more preferably greater than 10 mg/ml. The higher the solubility of the solute in the steady state solvent/anti-solvent composition, the more Ostwald ripening occurs, perhaps to particle sizes of up to 500 μm and even up to 1 mm.

Depending upon the prevailing supersaturation upon mixing, for a given solute, solvent and anti-solvent system, a high flow rate ratio, such as, for example, 400:1 to 1000:1 may be used to yield precipitative crystallization with very minimum growth, thereby leading to the formation of relatively small particles of around 100 nm to 100 μm. Conversely, for the same solute, solvent and anti-solvent system, a low flow rate ratio, such as, for example, 10:1 to 50:1 may be used to lead to the formation of relatively large particles of around 100 μm to 1 mm. The exact outcome will depend upon factors such as supersaturation, primary nucleation, secondary nucleation and crystal growth.

For this reason, depending upon the solute solubility in the resulting solvent/anti-solvent composition a modestly low ratio of say 50:1 or 10:1, and depending upon the ultrasonic power used, small particles could still be produced due to rapid dispersion and crystallization. The exact conditions will be substrate/solute dependent as determined by those skilled in the art.

In a further embodiment, the flow rate ratio is less than 1:1, such as less than 0.8:1 or less than 0.5:1. In this embodiment, the anti-solvent would be sufficiently powerful to mediate supersaturation and hence crystallisation would occur.

Typically, the flow rate of the anti-solvent stream through an UOBR apparatus suitable for producing crystalline particles using the process of the invention is preferably in the range of litres per hour (l/hr) [e.g. 20 l/hr] rather than millilitres per hour (ml/hr) and may be any flow rate suitable for the end purpose in question. In particular the combined flow rate of the solvent and anti-solvent through the UOBR will provide a residence time in the ultrasonic field in the range of 0.1 second to 1 hour, preferably 1 second to 30 minutes, more preferably 3 seconds to 3 minutes. For example, the flow rate for the first stream flow of the invention may be in the range 1 to 10 l/hr, preferably 3 to 7 l/hr, such as about 5 l/hr and that of the second stream flow may be in the range 0.1 to 1 Vhr, preferably 0.3 to 0.7 l/hr, such as about 0.5 l/hr for a bench top UOBR apparatus. Where the process is employed in a larger apparatus, for example, manufacturing scale, the throughput flow rates for the first stream may be in the range 100 to 1000 Vhr, preferably 30 to 700 l/hr, such as about 500 l/hr and for the second stream may be in the range 10 to 100 l/hr, preferably 30 to 70 l/hr such as about 50 l/hr. Naturally, the man skilled in the art will appreciate that the rate of flow for each of the said streams can be at any desired speed provided that the flow rate ratio of the two streams is that described for the present invention.

The flow rate of the anti-solvent, for a small scale UOBR apparatus, such as one having a 1 litre capacity, 5 litre or 10 litre capacity, may be up to 20 l/hr, typically up to 15 l/hr, up to 10 l/hr or up to 5 l/hr or of any value in between, such as 4 l/hr, 8 l/hr, 13/hr for example. The flow rate may be decided upon by the skilled addressee depending on the size of particles required for a chosen administration route to a site of interest for a particular end purpose. Correspondingly, the flow rate of the added solution of solute in solvent will be less than that of the anti-solvent with which it is to be placed in contact. An example of a flow rate ratio (20:1) used in the present invention is to be found in the examples wherein the anti-solvent flows at 0.6 l/hr and the solute in solvent at 60 m l/hr.

It will be appreciated that the anti-solvent and the solvent should be selected as being suitable for a particular substance or substances, such as at least one active principal or active precursor thereof. The selection of appropriate solvent and anti-solvent must be made in accordance with the physical properties of at least one substance to be crystallised.

The solute is a substance, typically an active principal or a desired precursor thereof, such as a drug or an agro-chemical of interest, which is able to form crystals in the process of the invention. There may be more than one solute comprised in the first flowing stream, for example, a mixture of two or more solutes of interest, such as two or more active principals of interest, for example, two or more drugs or two or more agro-chemicals, depending on the proposed end use of the said solutes.

The solutes and active principles listed in the specification include the salt and/or solvates thereof.

Turning to thermoregulated crystallization processes as a further embodiment to the present invention, temperature control is critical in crystallization processes, including fine and speciality chemicals, pharmaceuticals, bulk chemicals and the food industries. In particular, many such processes rely on the maintenance of a constant temperature, or a controlled decrease in temperature. The maintenance and control of temperature becomes particularly important in large batch scale crystallization processes.

However, in such large scale crystallization processes the degree to which temperature regulation, and in particular cooling or heating, can be controlled is troublesome due to the size of reactors and relatively poor heat transfer between the bulk of the liquid and walls of the reactor. This is extremely important in both crystallization, whereby supersaturation levels (as a function of temperature) should ideally be quite constant and without abrupt changes of supersaturation, and reactive chemistry for reasons of reaction selectivity and undesirable impurity formation and the like, as well as, control of exothermic heat output and endothermic heat input.

According to an aspect of the present invention, there is provided a process for preparing crystalline particles of at least one substance comprising subjecting a saturated or supersaturated flowing stream of at least one solute in solution to oscillatory motion in an oscillating baffled reactor; applying ultrasonic radiation; and collecting the particles that are generated. Preferably, the saturated or supersaturated stream is formed by heating or cooling the solvent stream. The person skilled in the art will understand that in many cases of forcing a solid phase out of solution the solubility of the particular substrate will decrease as the solution becomes cooler. Inverse solubility is the phenomenon in which a material becomes less soluble in a liquid as the temperature of the solution increases. In this case raising the temperature decreases solubility in turn leading to supersaturation. Inverse solubility is rare for organic molecules.

In the invention, the ultrasonic radiation is applied during at least a portion of the oscillatory motion. The oscillatory motion is applied during at least one portion of the ultrasonic motion. In the invention, the oscillating motion and the application of ultrasound both occur simultaneously for at least part of the process. Oscillatory motion and the application of ultrasound may additionally occur separately for part of the process. The ultrasonic radiation may also be applied throughout the duration of the application of the oscillatory motion. The oscillatory motion may be applied throughout the duration of the application of the ultrasound.

In a further embodiment, the saturated or supersaturated stream is formed by the process of the second aspect of the invention.

According to the present invention there is provided an oscillating baffled reactor apparatus for preparing crystalline particles of at least one substance comprising: a reactor vessel; means for supplying a first flowing stream; means for oscillating fluid within the reactor vessel; a plurality of baffles; source of ultrasonic; and means for collecting said particles. The apparatus preferably comprises a means for heating or cooling a flowing stream, preferably via one or more heat exchangers, preferably one or more liquid or air heat exchangers, attached to the vessel walls. Alternatively electrical heaters or refrigeration devices attached to the vessel walls can be used for heating and cooling respectively. The apparatus may further comprise a means for monitoring the temperature of the flowing streams such as a thermostat, a thermometer or the like. The apparatus may further comprise a means for testing for a saturated or supersaturated solution. Such process analytical tools available include immersed probes for measuring, for example, ultraviolet light or infra red light absorption and hence correlating the amount of absorption with prevailing solution concentration. The apparatus may have suitable inlets and outlets and pumping arrangements for process fluids such as a saturated solution of at least one substance, and may have a series of tubular units, each defining a discrete process zone, arranged and operatively connected in a flow system to form at least one continuous fluid flow path having an inlet and an outlet, wherein mixing means is provided within the flow path, and wherein each zone may have ultrasonic modules for effecting crystal nucleation and growth, dispersion, and disruption therein, and wherein each zone may have temperature regulating means for effecting temperature control therein.

Therefore, the vessel can be set such that the temperature of the contents is different in different process zones or flow paths, and moreover the contents are subject to ultrasonic radiation of defined intensity and time. This can be done accurately and consistently, giving greater control over the temperature of the contents and ultrasound effects upon the solid—liquid contents of the tubular units of the vessel. This is very useful in crystallization where the accurate control of temperature and ultrasound assisted nucleation, growth, dispersion and disruption have a significant impact on the end product. The preferred mixing means comprises of a plurality of baffles along with the localized mixing effects of the ultrasonic cavitation and shock waves.

Critical control of supersaturation is important in cooling crystallization processes because of the well known effects of rapid nucleation at high supersaturation which can lead to troublesome thick slurries. Alternatively one may use a powerful crystal nucleation tool, such as ultrasound, so as to bring about crystallization at relatively low supersaturation thereby avoiding the formation of the thick particle slurries potentially formed when a nucleation tool is not used. The present invention facilitates the synergistic benefits of improved bulk mixing by oscillatory flow, cavitational micromixing and crystal nucleation via the application of ultrasound. The apparatus may further comprise a means for testing for a saturated or supersaturated solution.

In another embodiment of the invention the flowing stream may contain a slurry of particles produced either by nucleation under other embodiments of this invention, or from a separate process. Particles produced in an earlier stage may not have the desired size range, surface morphology or crystallinity. The combination of the agitation of the OBR, which keeps the particle slurry well mixed, with the ultrasonic waves providing cavitation events, which impact on the particles provides an ideal mechanism for particle processing.

One action of cavitation provided by the ultrasonic waves can be used to break the particles into smaller particles, thus reducing the particle size range. A further effect of the ultrasonic waves is to aid the conversion of previously amorphous particles into more stable crystalline forms.

According to this aspect of the present invention there is provided an apparatus for controlling a process, comprising a vessel with suitable inlets and outlets and pumping arrangements for a process fluid, such as a slurry of particles suspended in an appropriate non-solvent, connected in a flow system to form at least one continuous fluid flow path having an inlet and an outlet, wherein mixing means is provided within the flow path, and wherein each zone may have ultrasonic modules for effecting the mechanical processing of the particles, and wherein each zone may have temperature regulating means for effecting temperature control therein.

Therefore the system can be used to provide ultrasonic processing of the suspended slurry to modify the particle size, morphology, stability and form to that desired of the process. By manipulating the solute solution flow-rate, cooling rate and ultrasonic treatment regime in the process of the present invention the inventors have now made it possible to provide crystals of active principals of interest of a desired size depending upon their end used and the solid-state chemistry in terms of nucleation and growth kinetics. The mean diameter size of particles that are able to be attained using the method of the invention lies in the range of from about 1 μm up to about 1000 μm. It will be appreciated that particle of around 1 μm to around 6 μm can be used for inhalation and oral suspension delivery whereby particles from around 10 μm to around 300 μm are more often used for tablets and capsules for oral delivery. Particles greater than around 300 μm will usually require some form of milling prior to drug formulation.

As shown above, a saturated or supersaturated solution of a solute in a solvent may be formed by either mixing with an anti-solvent stream or by heating and cooling, or both. This saturated or supersaturated solution may form crystalline particles in the presence of ultrasound.

Suitable solutes that are able to crystallise under the process conditions of the invention include active principals or drugs or salts thereof which can be formed into crystalline particles by the process of the present invention such as corticosteroids, β2-agonists, anticholinergics, leukotriene antagonists, inhalable proteins or peptides, mometasone furoate; beclomethasone dipropionate; budesonide; fluticasone; dexamethasone; flunisolide; triamcinolone; salbutamol; albuterol; terbutaline; salmeterol; bitolterol; ipratropium bromide; oxitropium bromide; sodium cromoglycate; nedocromil sodium; zafirlukast; pranlukast; formoterol; eformoterol; bambuterol; fenoterol; clenbuterol; procaterol; broxaterol; (22R)-6a,9a-difluoro-llb,21-dihydroxy-16a,17a-propylmethylenedioxy-4-pregnen-3,20-dione; TA-2005; tipredane; insulin; interferons; calcitonins; parathyroid hormones; granulocyte colony-stimulating factor and mixtures of two or more thereof.

Other particles which may be made according to the invention include any drugs or active principals usefully delivered by inhalation for example, analgesics, e.g. codeine, dihydromorphine, ergotamine, fentanyl or morphine; anginal preparations, e.g. diltiazem; antiallergics, e.g. cromoglycate, ketotifen or nedocromil; anti-infectives, e.g. cephalosporins, penicillins, streptomycin, sulphonamides, tetracyclines or pentamidine; antihistamines, e.g. methapyrilene; anti-inflammatories, e.g. beclomethasone, flunisolide, budesonide, tipredane, triamcinolone acetonideor fluticasone antitussives, e.g. noscapine; bronchodilators, e.g. ephedrine, adrenaline, fenoterol, formoterol, isoprenaline, metaproterenol, phenylephrine, phenylpropanolamime, pirbuterol, reproterol, rimiterol, salbutamol, salmeterol, terbutalin; isoetharine, tulobuterol, orciprenaline or (−)-4-amino-3,5-dichloro-a[[[6-[2-(2-pyridinyl)ethoxy]hexyl]amino]methyl]benzenemethanol; diuretics, e.g. amiloride; anticholinergics e.g. ipratropium, atropine or oxitropium; hormones, e.g. cortisone, hydrocortisone or prednisolone; xanthines e.g. aminophylline, choline theophyllinate, lysine theophyllinate or theophylline; and therapeutic proteins and peptides, e.g. insulin or glucagon and glycopyrronium bromide.

Preferably, the crystalline particles are a pharmaceutically active ingredient selected from the group consisting of anti-allergics, bronchodilators, anti-inflammatory steroids antibiotics, antivirals, antiinfectives, oncolytics, pain management medicines, Central Nervous System medicines, cardiovascular medicines, Parkinson disease medicines, HIV antivirals, epilepsy medicines, gastrointestinal medicines, musculoskeletal medicines, medicines for metabolic disorders, genitor-urinary medicines and orthopedic medicines and mixtures thereof.

It will be appreciated by the person skilled in the art that, where appropriate, medicaments comprising active principals or drugs may be used in the form of salts (e.g. as alkali metal or amine salts or as acid addition salts) or as esters (e.g. lower (C₁₋₇) alkyl esters) or as solvates (e.g. hydrates) to optimise the activity and/or stability of the medicament.

Particularly suitable medicaments for preparation with particles obtained in accordance with the process of the invention include anti-allergics, bronchodilators and anti-inflammatory steroids of use in the treatment of respiratory disorders such as asthma by inhalation therapy, for example cromoglycate (e.g. as the sodium salt), salbutamol (e.g. as the free base or as the sulphate salt), salmeterol (e.g. as the xinafoate salt), terbutaline (e.g. as the sulphate salt), reproterol (e.g. as the hydrochloride salt), beclomethasone dipropionate (e.g. as the monohydrate), fluticasone propionate or (−)-4-amino-3,5-dichloro-α-[[[6-[2-(2-pyridinyl)ethoxy]hexyl]amino]-methyl]benzenemethanol and physiologically acceptable salts and solvates thereof.

The present invention relates to the physical process of crystallising a substance or substances to form small crystals or small co-crystals such as 500 nm to 10 μm, preferably 600 nm to 5 μm, most preferably 650 nm to 2 μm, when the means of drug delivery is by inhalation or aqueous oral suspension. The present invention also relates to the physical process of crystallising a substance or substances to form crystals or co-crystals such as 2 μm to 900 μm, preferably 5 μm to 400 μm, most preferably 10 μm to 200 μm when the means of drug delivery is oral tablet or capsule.

The invention preferably does not comprise reactive chemistry, that is whereby the resulting small crystals or co-crystals are made by reaction of one solute with at least one other component in the reaction system; such as another solute, an insoluble substance, a solvent or an anti-solvent; to form a reactive product.

It will be appreciated by the person skilled in the art that particles made by the process of the invention may contain a combination of two or more active principals and these two or more active principals may form co-crystals. Active principals may be selected from suitable combinations of the active principals mentioned hereinbefore. Thus, suitable combinations of bronchodilatory agents include ephedrine and theophylline, fenoterol and ipratropium, and isoetharine and phenylephrine.

Further suitable combinations of particles of active principals made according to the process of the invention include combinations of corticosteroids, such as budesonide, beclomethasone dipropionate and fluticasone propionate, with 132-agonists, such as salbutamol, terbutaline, salmeterol and formoterol and physiologically acceptable derivatives thereof especially salts including sulphates.

Other examples of particles obtainable by the process of the invention may include a cromone which may be sodium cromoglycate or nedocromil, or a carbohydrate, for example, heparin.

The particles made by the process of the invention may comprise an active principal suitable for inhalation and may be a pharmacologically active agent for systemic use. For example, such active particles may comprise peptides or polypeptides or proteins such as Dase, leukotines or insulin (including pro-insulins), cyclosporin, interleukins, cytokines, anticytokines and cytokine receptors, vaccines, growth hormone, leuprolide and related analogues, intereferons, desmopressin, immunoglobulins, erythropoietin and calcitonin.

Alternatively, the active principal made by the process of the invention may be suitable for oral administration. A drug for oral administration may be one of the systemic drugs mentioned above. The active principal may be a substance which exhibits low solubility in the digestive tract, for example, magnesium trisilicate, calcium carbonate and bismuth subnitrate. Organic compounds may include, for example, all products of combinatorial chemistry, rosiglitazone and other related glitazone drugs, hydrochlorothiazide, griseofulvin, lamivudine and other nuclease reverse transcriptase inhibitors, simvastatin and other statin drugs, benzafibrate and other fibrate drugs and loratidine, and any other physiologically tolerable salts and derivatives thereof.

Pharmaceutical excipients suitable for adding to particles made according to the process of the invention include, for example, carbohydrates especially monosaccharides such as fructose, glucose and galactose; non-reducing disaccharides such as sucrose, lactose and trehalose; non-reducing oligosaccharides such as raffinose and melezitose; non reducing starch derived polysaccharides products such as maltodextrins, dextrans and cyclodextrins; and non-reducing alditols such as mannitol and xylitol. Further suitable excipients include cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone (PVP). Mixtures of two or more of any of the above excipients are also envisaged.

Where the particles of active principal(s) prepared by the process of the present invention are agro-chemically active, the active principal may for example be a plant growth regulator, herbicide, and/or pesticide, for example insecticide, fungicide, acaricide, nematocide, miticide, rodenticide, bactericide, molluscicide or bird repellent.

Examples of organic water-insoluble agrochemical active principals made according to the process of the invention include insecticides, for example selected from the group consisting of carbamates, such as methomyl, carbaryl, carbofuran, or aldicarb; organo thiophosphates such as EPN, isofenphos, isoxathion, chlorpyrifos, or chlormephos; organo phosphates such as terbufos, monocrotophos, or terachlorvinphos; perchlorinated organics such as methoxychlor; synthetic pyrethroids such as fenvalerate; nematicide carbamates, such as oxamyl herbicides, for example selected from the group consisting of triazines such as metribuzin, hexaxinone, or atrazine; sulfonyl ureas such as 2-chloro-N-[(4-methoxy-6-methyl-l,3,5-triazin-2-yl)aminocarbonyl]-benzenesulfonamide; uracils (pyrimidines) such as lenacil, bromacil, or terbacil; ureas such as linuron, diuron, siduron, or neburon; acetanilides such as alachlor, or metolachlor; thiocarbamates such as benthiocarb (SATURN), triallate; oxadiazol-ones such as oxadiazon; phenoxyacetic acids such as 2,4-D; diphenyl ethers such as fluazifop-butyl, acifluorfen, bifenox, or oxyfluorfen; dinitro anilines such as trifluralin; glycine phosphonates such as glyphosate salts and esters; dihalobenzonitriles such as bromoxynil, or ioxynil; fungicides, for example selected from the group consisting of nitrilo oximes such as cymoxanil (curzate); imidazoles such as benomyl, carbendazim, or thiophanate-methyl; triazoles such as triadimefon; sulfenamides such as captan; dithiocarbamates such as maneb, mancozeb, or thiram; chloronated aromatics such as chloroneb; dichloro anilines such as iprodione; aphicides, for example selected in the group consisting of carbamates, such as pirimicarb; miticides, for example selected from the group consisting of propynyl sulfites such as propargite; triazapentadienes such as amitraz; chlorinated aromatics such as chlorobenzilate, or tetradifan; and dinitrophenols such as binapacryl.

The organic water-insoluble agrochemical active principals may be comprised in the particles produced according to the present invention as a mixture of several ingredients. Especially preferred organic water-insoluble agrochemical active ingredients are atrazine, cymoxanil, chlorothalanil, cyproconazole, and tebuconazole.

When more than one solute is used, co-crystals may be formed. Co-crystals can be defined as crystalline complexes of two or more non-identical neutral molecular constituents, such as an active principal or desired precursor thereof, and a guest bound together in the crystal lattice through noncovalent interactions, preferably primarily hydrogen bonding. A guest may be another active principal or desired precursor thereof, or a co-crystal former.

The formation of pharmaceutical co-crystals involves incorporation of a given active pharmaceutical with another pharmaceutically acceptable molecule in the crystal lattice. The resulting multi-component crystalline phase will maintain the intrinsic activity of the parent active pharmaceutical while possessing a distinct physiochemical profile.

As used herein, the term “co-crystal former” denotes one or more additional molecules present in the same crystal structure as the active principal, or desired precursor thereof, which one or more additional molecules are capable of forming a supramolecular synthon with the active principal, or desired precursor thereof, by way of the intermolecular interactions characteristic of the bonding in a co-crystal.

In one embodiment, the co-crystal former comprises one or more molecules having at least one synthon forming moiety selected from the following group: ether, thioether, alcohol, carbonyl, thiol, aldehyde, ketone, thioketone, nitrate ester, phosphate ester, thiophosphate ester, ester, thioester, sulphate ester, carboxylic acid, phosphonic acid, phosphinic acid, sulphonic acid, sulphonamide, amide, primary amine, secondary amine, ammonia, tertiary amine, imine, thiocyanate, cyanamide, oxime, nitrile, diazo, organohalide, nitro, S-containing heterocyclic ring (such as thiophene), N-containing heterocyclic ring (such as pyrrole, imidazole or pyridine), O-containing heterocyclic ring (such as furan, epoxide or peroxide) and hydroxamic acid moieties.

In further embodiments, the guest may be present, for example, in order to form the co-crystal with the active principal or desired precursor thereof. It is contemplated that one or more guests may be included in a co-crystal. Accordingly, the guest is not required to have an activity of its own, although it may have some activity that does not overly derogate from the desired activity of the active agent. A non-active guest may be a compound where no beneficial pharmacological activity has been demonstrated and which are appreciably biologically non-toxic or pharmacologically benign. In some situations, the guest may have the same activity as or an activity complementary to that of the active agent. The guest may be another active principal or desired precursor thereof. For example, some guests may facilitate the therapeutic effect of an active principal or desired precursor thereof. For pharmaceutical formulations, the guest may be any pharmaceutically acceptable molecule(s) that form a co-crystal with the active principal or desired precursor or its salt.

The guest, or co-crystal former, may be an acid and behave in both a neutral manner but with noncovalent interactions (primarily hydrogen bonding), such as in the case of oxalic acid or other suitable carboxylic acids when prepared as a co-crystal with caffeine, and as a proton-donor when in the case of forming ionic salts such as in the reaction or proton-exchange with an amine for example. Similarly benzoic acid and succinic acid behave in a neutral manner (without formal proton exchange) when forming a co-crystals with fluoxetine hydrochloride or in a proton-exchange manner to form ionic salts such as sodium benzoate or sodium succinate. These compounds may be ionic guests in their own right. Neutral guests are preferably nonionic guests. Ionic guests are compounds or complexes having ionic bonding. The guest may be an acid that forms hydrogen bonds with the chloride (or other anion). Ionic guests are compounds or complexes having ionic character, as exemplified by ionic interaction and attraction. The guest may be an acid that forms hydrogen bonds with the pharmaceutical ingredient. For example, suitable guests which are acids include (but not are not limited to): ascorbic acid, glucoheptonic acid, sebacic acid, alginic acid, cyclamic acid, ethane-1,2-disulfonic acid, 2-hydroxyethanesulfonic acid, 2-oxo-5 glutaric acid, naphthalene-1,5-disulfonic acid, nicotinic acid, pyroglutamic acid and 4-acetamidobenzoic acid. The solutes and active principles listed in the specification include the salt and/or solvates thereof. Co-crystals are described in WO2005/089375.

An example of a co-crystal of the present invention is sildenafil, or a pharmaceutically acceptable salt thereof, and acetylsalicylic acid (aspirin).

In one embodiment, the active principal or desired precursors thereof is the solute in the first flowing stream and is insoluble in the second flowing stream; and the guest may be soluble in the first and second flowing stream; or insoluble in the first and second flowing stream; or soluble in one of the first and second flowing streams and insoluble in the other stream. In these embodiments, a co-crystal may be formed.

In a further embodiment, the guest is the solute in the first flowing stream and is insoluble in the second flowing stream and the active principal or desired precursor thereof may be soluble in the first and second flowing stream; or insoluble in the first and second flowing stream; or soluble in one of the first and second flowing streams and insoluble in the other stream. In these embodiments, a co-crystal may be formed.

According to the process of the third aspect of the invention, the saturated or supersaturated flowing stream may comprise two solutes, and co-crystals may be formed.

It will be appreciated that the anti-solvent and the solvent should be selected as being suitable for a particular active ingredient or active precursor thereof. Corticosteroids, such as budesonide, beclomethasone dipropionate and fluticasone propionate may be dissolved in dichloromethane or methanol and ultrasonically treated in anti-solvents such as heptane. β-agonists, such as salmeterol, xinafoate and formoterol fumarate, may be dissolved in methanol and ultrasonically treated in anti-solvents such as acetone, ethyl acetate or heptane.

In a further embodiment the single of multiple components may be dissolved with heating in a suitable solvent or solvent composition until a saturated solution is obtained. This solution may then be fed into a suitable OBR configured with temperature and ultrasound management as previously described. The solution, once cooled to some extent, will become supersaturated and thus in a labile state whereupon crystallization of single or multiple substrates may occur and indeed a co-crystal may form from a homogeneous solution of the substrate and the co-crystal former.

As described above, the apparatus is an oscillating baffled reactor apparatus for preparing crystalline particles of at least one substance comprising: a reactor vessel; means for supplying a first flowing stream; means for oscillating fluid within the reactor vessel; a plurality of baffles; source of ultrasonic; and means for collecting said particles. In one embodiment, the first flowing stream comprises a solution of at least one solute in a solvent; wherein the apparatus further comprises a means for supplying a second flowing stream comprising an anti-solvent.

As described above, the apparatus may be an apparatus for preparing crystalline particles of at least one substance comprising a reactor vessel; means for supplying a first flowing stream comprising a solution of at least one solute in a solvent; means for supplying a second flowing stream comprising an anti-solvent; means for oscillating fluid within the reactor vessel; a plurality of baffles; source of ultrasonic radiation; and means for collecting said particles

In embodiments, the apparatus comprises an ultrasonic oscillatory baffled reactor, (UOBR), which comprises an elongated tubular vessel having inlets at one end for the liquid feeds, outlet at the other end and at least one ultrasonic module, preferably more than one ultrasonic module, positioned along the length of the elongated vessel; the elongated vessel being provided with means for imposing on the liquids within the vessel oscillatory motion in a defined direction, and a plurality of obstacles i.e. baffles, substantially transverse to the direction of liquid flow. Preferably the baffles are stationary baffles. In one embodiment the source of ultrasonic radiation is at least one ultrasonic section arranged along the reactor vessel.

The baffles can be spaced on the inner walls of the vessel and can be mounted at intervals. Preferably the baffles have sharp edges. For example they can have a thin rectangular, triangular or diamond lateral cross-section. In one embodiment, the baffles will be in the form of flat discs having substantially concentric annular holes which look like washers spaced along the inside of the vessel. The axial spacing between adjacent baffles i.e. between adjacent rings, can be from 0.5 to 4 times the diameter of the tubular vessel, preferably about 1.5 times the diameter.

Increasing the baffle area and reducing the space between the baffles increases the Reynolds number of the fluid and increases the localised turbulence within the reactor vessel. Increased turbulence and an increased Reynolds number result in smaller particles forming. Decreased turbulence and a decreased

Reynolds number result in larger particles forming. A high Reynolds number contributes to efficient dispersion, mixing, nucleation and small particle formation.

The Reynolds number of the oscillatory motion set up between adjacent baffles is desirably above 100 and preferably in the range 200-300 or above, and often over 2000, and where a unidirectional motion through the vessel is superimposed on the essential oscillatory one, the Reynolds number of the unidirectional flow is preferably less than the peak of the Reynolds number of the oscillatory motion

The oscillatory pulsing frequency and amplitude, coupled with both axial and radial mixing will lead to superior mixing within individual segmented (baffled) zones. The effects of extremely good mixing will be to smooth out supersaturation peaks in local regions, particularly micromixing, that is, mixing at the near molecular level, which will lead to consistent and generally smaller particles, either by interparticulate collision or nucleation, which is also helped by ultrasound, at the expense of crystal growth. In addition where there is a separate feed into the tubular reactor, meso-mixing, that is, implied turbulent dispersion when one solution meets another.

The liquids entering by the inlets causes a net and unidirectional flow along the vessel and the product crystalline slurry is withdrawn from the outlet. Superimposed on this flow is oscillatory motion, so that liquid flow reversal takes place. Preferable the oscillatory motion is axial oscillation motion. The velocity of the liquid or liquid slurry in the general direction rises and falls in a continuous manner and passes through a negative value whilst retaining a net flow along the tubular reactor. The oscillatory flow can be provided by pumping the liquid using a centrifugal or gear pump, and providing reciprocating motion by use of suitable pumps, valves or mechanisms. Pistons suitably located can provide mechanism for providing oscillatory flow. Concurrent with the superimposition of oscillatory motion, ultrasonic modules are positioned close to the reciprocating pump or at various intervals along the reactor in order to provide a means of ultrasound assisted dispersion and crystal nucleation. Such nucleation can be at the expense of crystal growth.

Preferably the pulsation of the oscillatory flow has an amplitude of 0.02×d−1.00×d, preferably 0.05×d−0.5×d, particularly preferably 0.10×d−0.2×d and very preferably of 0.13×d (+−0.2), where d is the internal diameter of the reaction vessel.

The frequency of the pulsation of the oscillatory flow may be in the range from 0.5 to 50 Hz, preferably from 1 to 10 Hz and particularly preferably from 6.5 Hz (+−3 Hz).

Generally, the more vigorous the pulsation, the more mixing that occurs, which results in better dispersion and smaller particles. A person skilled in the art is able to select a suitable pulsation amplitude and frequency for a given solute, solvent and anti-solvent system.

The invention can be described by way of example, making reference to FIGS. 1-6. The UOBR comprises an elongated reactor section (3), ultrasonic modules (6, 34, 41), reciprocating pump (2) and baffles (10).

Inlets are associated with one end of the reactor, located appropriately close to the pump that provided oscillatory motion to the liquid medium. The fluid is oscillated in the axial direction by means of diaphragms, bellows or pistons at one or both ends of the tube.

Baffles are provided along the length of the reactor whereby each baffle comprises a ring which is attached either to the inner wall of the reactor section or suitable baffle supports.

The ultrasonic modules or chambers are positioned as appropriate at various positions along the length of the reactor or alternatively a single module is positioned very close the pump itself.

The flowing stream of solvent comprising solute (i.e. the ‘solution’) and the flowing stream of anti-solvent may be contacted or mixed together such that the two streams flow along a single path or axis in the same direction, for example, within the lumen of a suitable delivery means and into a suitable receptacle or chamber, such as an ultrasonic continuous flow cell. Each of the said flowing streams may be pumped at a pre-determined rate of flow from an initial source reservoir into the delivery means.

Preferably the solvent and the anti-solvent are delivered into the reactor is parallel contacting streams. The anti-solvent and solvent inlets are adjacent to each other such that the streams of liquid outflowing from each inlet contact along one side of the stream in one embodiment.

A suitable delivery means for the first and second flowing stream, where required, may comprise a tubular means such as a straight or curved conduit, for example a pipe. When there are two flowing streams, the two streams may be mixed coaxially therein. Alternatively, the two streams may be introduced into the ultrasonic continuous flow cell module, via pumping through separate delivery means, such as two separate tubular means, for example, two pipes. Coaxial delivery is preferred since it brings about intimate mixing in the ultrasound and localised turbulence.

In a further embodiment, the anti-solvent and the solvent streams enter the UOBR at different positions to avoid localised concentrations of anti-solvent in the vicinity of the solute/solvent inlet.

For temperature managed processes, the flowing stream of heated/cooled solution of solute in solvent (i.e. the ‘solution’) may be passed along a single path or axis, for example, within the lumen of a suitable delivery means and into a suitable receptacle or chamber, such as an ultrasonic continuous flow cell.

The solution may be pumped at a pre-determined rate of flow from an initial source reservoir into the delivery means.

Once inside the UOBR the combined streams of anti-solvent and solvent for antisolvent processing or heated/cooled solution for temperature managed processing are subjected to ultrasonic irradiation, oscillatory motion and plug-flow behaviour to form crystals of a desired mean size. The ultrasonic energy induces nucleation and subsequent crystallisation of the solute in the anti-solvent in the operating vicinity of the ultrasonic energy, or of an ultrasonic energy transducer, such as a device with a multiplicity of ultrasonic transducers placed circumferentially around a cylindrical duct, if such a configuration is employed. The ultrasonic energy may be applied continuously or in a discontinuous manner, such as by pulsed application. Any suitable source of ultrasonic irradiation may be used. The sources of ultrasound may use either piezoelectric or magnetorestrictive devices. These include an ultrasonic probe, for example, inserted into a mixing vessel, such as a continuous ultrasonic flow cell; an ultrasonic emitter contained within the mixing vessel; the mixing vessel may be housed in an ultrasonic bath or a multiplicity of ultrasound transducer fixed to the walls of the mixing vessel, preferably the external walls of the mixing vessel.

The amplitude and frequency of the ultrasound waves affects the rate of nucleation and crystal growth. Single or multiple frequencies of ultrasound waves may be applied either continuously or in pulsed modes depending on process requirements. The frequency of the ultrasound waves may for example be from 10 kHz to 1 MHz, preferably from 10 to 500 kHz, more preferably from 10 to 100 KHz such as at 10, at 20, 40, 60, 80, or 100 KHz or at any frequency thereinbetween, such as, 20 kHz or 40 kHz. The pulsing frequency of oscillation affects the resulting particle size of the crystalline particles. Increasing the oscillation rate will increase the Reynolds number, increase the localised turbulence which will lead to reduced particle size of the resulting crystalline particles.

The ultrasonic irradiation is employed at an amplitude that is appropriate for the formation of crystals of the desired size, for a pre-determined application. For laboratory probe systems with an emitting face of, for example 80 cm², the amplitude selected may be from about 1-30 μm, typically from 3-20 μm, preferably from 5-10 μm, for example, 5 μm. Probes having a probe face surface area of 8 cm² and a power requirement of from 5-80 W, provide a power density of from 0.6-12.5 W/cm² using an amplitude of 2-15 μm. In larger systems, comprising transducers bonded onto the flow cell, for example a 2 litre flow cell, the power density for the transducers employed may be from 50-200 W/I, preferably from 80-600 WA and more preferably from 100-200 W/I, for example 120 W/1 or 200 W/I.

An increase in ultrasound amplitude will result in increased cavitations in the localised region. This cavitational energy input has a dramatic effect upon crystal nucleation at relatively low supersaturation. This can lead to a dramatic increase in the number of crystal nuclei, and hence number of crystals at the end of the process, and therefore leading to smaller particle size distribution.

The residence time of the mixed components in each ultrasonic flow cell may be from 0.1 s up to about 10 s or longer. For continuous systems the residence time can be longer depending on design and may be several minutes. The skilled addressee will appreciate that the residence time in the ultrasonic flow cell for each volume of fluid that is placed in it will be of the order of 0.1 s up to 10 s, depending on design.

Longer residence time may be required at lower supersaturation. This depends upon feed rate of input solution or reagent. The longer the residence time, the more cavitations, which results in more crystals when compared with a shorter residence time.

If there is more than one ultrasound module, the second and subsequent modules can be used to encourage further primary and secondary nucleation and possibly particle attrition further down the tubular reactor.

The total residence time in the reactor vessel can range from 0.1 seconds to 1 hour, preferably 1 second to 30 minutes, more preferably 10 seconds to 3 minutes.

The process may be employed in OBR reactors employed in the art such as a continuous OBR flow reactor, depending on design. The man skilled in the art is well acquainted with such reactor types and their operation. Generated crystals may be gathered or harvested from the UOBR by drawing off crystals using conventional means in the art, such as by the process described in WO03/092851. These means include filtration, spray-drying, supercritical carbon dioxide drying, and lyophilisation when water is used.

An advantage of using an OBR is that the flow rate approaches plug-flow chemistry, therefore the OBR can be seen to consist of a theoretical number of different reaction zones. In ideal plug-flow there is no mixing in the axial direction and there is complete mixing the radial direction.

The invention will now be described with reference to the accompanying examples and figures. It is to be understood that the examples and figures are not to be construed as limiting the scope of the invention in any manner.

The process of the invention may be carried out using OBR equipment as shown in the accompanying figures in which:

FIG. 1 shows a longitudinal sectional view of a crystallisation apparatus incorporating two separate feed stream delivery means for the solvent and anti-solvent leading into an ultrasonic continuous flow cell configured as UOBR having an ultrasonic transducer assembly replaced therein;

FIG. 2 shows a longitudinal sectional view of a crystallisation apparatus which is configured similarly to the apparatus in FIG. 1 except that a multiplicity of ultrasonic modules/flow cells (6) are positioned at various intervals along the tubular OBR.

Turning to FIG. 1, continuous UOBR crystallisation apparatus 1 comprises a means to provide oscillatory flow motion by way of pump 2 through the tubular reactor 3 through which liquid anti-solvent flows into a delivery means 4 and is pumped at a first flow rate via pump 5 into an ultrasonic flow cell chamber 6. Concurrently, a liquid solute in solvent is pumped via a pump 7 at a flow rate different from that of the anti-solvent via delivery means 8 and into ultrasonic flow cell chamber 6 where the two liquids are mixed under the influence of oscillatory motion. The “back and forth” oscillatory flow is provided by a suitable means known to a person skilled in the art. These means include a reciprocating pump or oscillating piston. Ultrasonic transducer 9 irradiates the mixture with ultrasonic energy and the mixture flows in an oscillatory action through the tubular reactor 3 in turn fitted with disc baffles 10. Thus in use of the apparatus 1, the saturated solution is thoroughly and rapidly mixed with the anti-solvent, the volume of the ultrasonic flow-cell 6 and the flow rates being such that the residence time in the ultrasonic flow cell chamber 6 is for example, 10 s. The ultrasonic energy from the transducer 9, allied with the oscillatory motion, insonates the entire volume of the flow cell 6 with sufficient intensity to cause dispersion and nucleation, as localised cavitation occurring on a microscopic scale promotes changes in fluid temperature and pressure that induce nucleation (and also promotes formation of the stable polymorph). By adjusting the power of the ultrasound, and the residence time in flow cell 6, the degree of nucleation can therefore be controlled. The ultrasound has the additional benefit that any crystal deposits within the flow cell 6 tend to be removed from the surfaces. The process slurry is passed through the tubular reactor 3 whereby oscillatory motion reduces the effects of crystal growth. The length of the tubular reactor 3 and flow rates can be modified so as to give the desired residence time and hence desired crystal size of the particles suspended in the slurry emerging from the end of the tubular reactor 3. The tubular reactor length maybe sensibly modified by means of U section baffles sections 10.

Turning to FIG. 2, continuous UOBR apparatus 20 is configured similarly to continuous UOBR apparatus 1 except that a multiplicity of ultrasonic modules/flow cells 6 are positioned at various intervals along the tubular OBR.

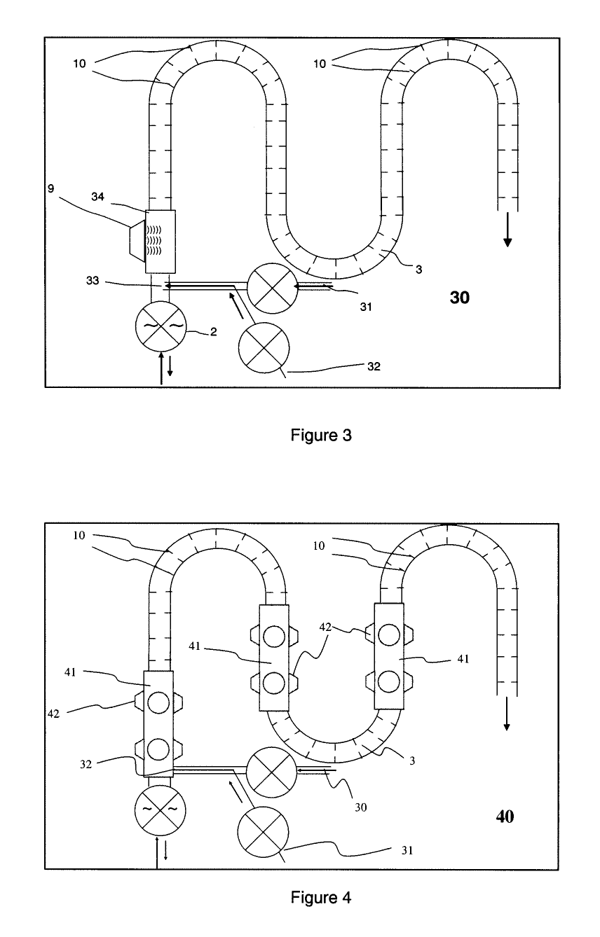

Turning to FIG. 3, and in contrast to the configuration of FIG. 1, continuous UOBR apparatus 30 is configures with the two liquid feeds from delivery means 31 and 32 and are contacted coaxially within a single delivery means 33 and fed into the ultrasonic flow cell chamber 34 via a single inlet.

Referring to FIG. 4, continuous UOBR apparatus 40 is of a similar configuration to that of FIGS. 1, 2 and 3 except that flow cell 41 has a multiplicity of ultrasonic transducers 42 placed circumferentially around a cylindrical duct. The transducer array 42 insonates the entire volume of the flow cell 41 with sufficient intensity to cause nucleation and by adjusting the power of the ultrasound, and the residence time in the flow cell 41, the degree of nucleation can therefore be controlled. The ultrasound has the additional benefit that any crystal deposits within the flow cell 41 tend to be removed from the surfaces. The flow cell can be positioned close to the inlet of the UOBR or at regular intervals along the UOBR as shown in FIG. 4.

The skilled addressee will again appreciate that the delivery means to the ultrasonic flow chamber 41 could also follow the configuration of that of FIGS. 1 and 2.

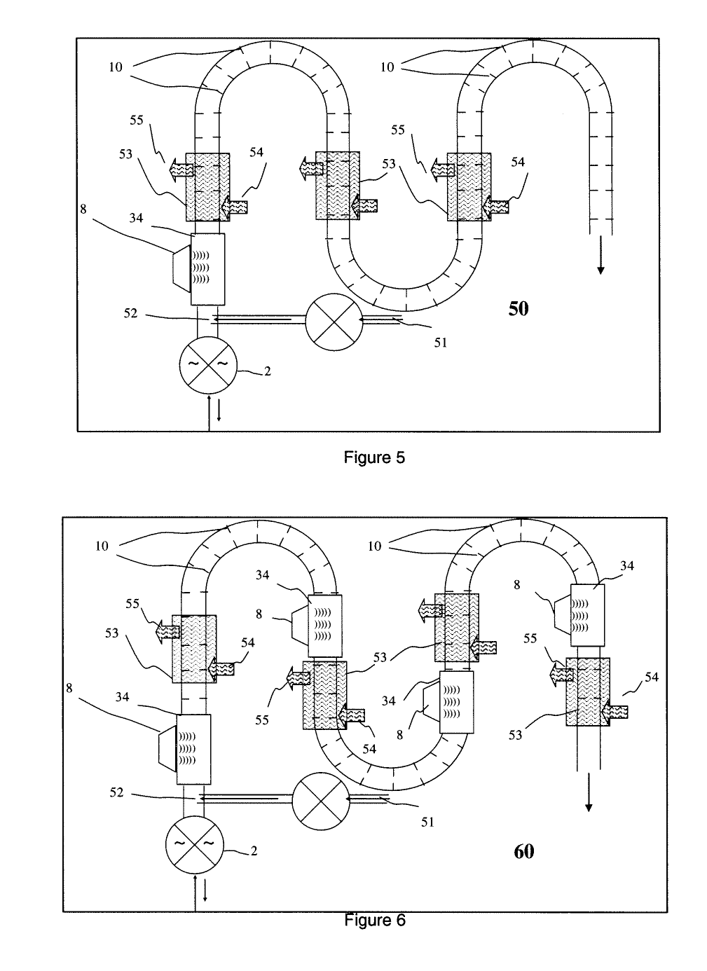

Turning to FIG. 5, continuous UOBR apparatus 50 is configured with a single delivery means 51 for introduction of supersaturated liquor into the ultrasonic continuous flow cell chamber 34 via a single inlet 52, configured as UOBR having an ultrasonic transducer assembly 8 and 34 positioned immediately after introduction of the liquor. The introduced liquor can be treated with ultrasound accordingly and then be subject to a temperature change via heat exchangers 53, with a heating or cooling medium introduced via inlet 54 and exiting by outlet 55, either with a single heat exchanger or a multiplicity thereof positioned along the UOBR.

Turning to FIG. 6, continuous UOBR apparatus 60 is configured with a single delivery means 51 for introduction of supersaturated liquor into the ultrasonic continuous flow cell chamber 34 via a single inlet 52, configured as UOBR having an ultrasonic transducer assembly 8 and 34 positioned immediately after introduction of the liquor and at various places along the UOBR. The introduced liquor can be treated with ultrasound accordingly and then be subject to a temperature change via heat exchangers 53, with a heating or cooling medium introduced via inlet 54 and exiting by outlet 55, either with a single heat exchanger or a multiplicity thereof positioned along the UOBR. The multiplicity of ultrasonic transducer assemblies 8 and 34 positioned along the UOBR facilitates further primary and secondary nucleation due to increase in supersaturation (as a function of the change in temperature), material disruption, restriction of crystal/particle growth, leading to particle uniformity.

In one particular configuration an alternation of heat exchanger and ultrasonic transducer assemblies can be used to provide continuous nucleation as a solution is passed through a temperature gradient generating increased supersaturation.

In a further configuration an pairs of heat exchangers can be used in conjunction with ultrasonic transducer assemblies, as in FIG. 6, to provide temperature cycling whereby levels of supersaturation is decreased then increased to aid dissolution of fine particles.

1. An oscillating baffled reactor apparatus for preparing crystalline particles of at least one substance comprising:
a reactor vessel;
means for supplying a first flowing stream;
means for oscillating fluid within the reactor vessel;
a plurality of baffles;
source of ultrasonic radiation; and
means for collecting said particles.

2. The apparatus according to claim 1, wherein the first flowing stream comprises a solution of at least one solute in a solvent; wherein the apparatus further comprises a means for supplying a second flowing stream comprising an anti-solvent.

3. The apparatus according to claim 1, further comprising a means for heating or cooling a flowing stream, preferably via one or more heat exchangers.

4. The apparatus according to claim 1, wherein the source of ultrasonic radiation is at least one ultrasonic transducer assembly, whereby a single transducer is attached to the wall of the assembly, arranged along the reactor vessel, preferably attached to the external wall of the assembly.

5. The apparatus according to claim 1, wherein the first flowing stream contains a slurry of particles.

6. The apparatus according to claim 1, wherein the source of ultrasonic radiation is an ultrasonic probe.

7. The apparatus according to claim 1, wherein the source of ultrasonic radiation is a wrap around ultrasonic transducer.

8. The apparatus according to claim 1, wherein the means for supplying the solution and the means for supplying the anti-solvent are oriented such that the solution and the anti-solvent are delivered into the reactor vessels in parallel contacting streams.

9. The apparatus according to claim 1, wherein the means for supplying the solution and the means for supplying the anti-solvent are via two separate inlets into the reactor vessel.

10. The apparatus according to claim 1, wherein the reactor vessel is a tubular vessel.

11. The apparatus according to claim 9, wherein the source of the ultrasonic radiation is a multiplicity of ultrasonic transducers attached circumferentially to the wall of the tubular vessel, preferably to the external wall of the tubular vessel.

12. The apparatus according to claim 1, wherein the longitudinal spacing between adjacent baffles is 0.5 to 4 times the diameter of the tubular vessel, preferably 1.5 times the diameter of the tubular vessel.

13. The apparatus according to claim 1, further comprising a means for testing for a saturated or supersaturated solution.

14. A process for preparing crystalline particles of at least one substance comprising contacting a solution of at least one solute in a solvent in a first flowing stream with an anti-solvent in a second flowing stream; subjecting the contacted streams to oscillatory motion in an oscillating baffled reactor; applying ultrasonic radiation; and collecting the particles that are generated.

15. The process of claim 14, wherein the frequency of the ultrasound waves is 10 kHz to 1 MHz, preferably 10 to 500 kHz, most preferably 10 to 100 kHz.

16. The process according to claim 14, wherein a flow rate ratio of anti-solvent:solvent is greater than 1:1, preferably the flow rate ratio is between 20:1 and 1000:1, more preferably 20:1 to 900:1, even more preferably 20:1 to 500:1, most preferably 20:1 to 400:1.

17. The process according to claim 14, wherein a flow rate ration of anti-solvent:solvent is less than 1:1, preferably less than 0.8:1, more preferably less than 0.5:1.

18. The process according to claim 14, wherein the anti-solvent and solvent are the same liquid at different temperatures, wherein the temperature of the anti-solvent and solvent lies between −10° C. and +120° C. and the temperature of the solvent and anti-solvent are separated by a temperature difference of at least 20° C., preferably at least 30° C., most preferably at least 50° C.

19. The process according to claim 14, wherein the anti-solvent and solvent are miscible liquids.

20. The process according to claim 14, wherein the anti-solvent and solvent are immiscible liquids.

21. A process for preparing crystalline particles of at least one substance comprising subjecting a saturated or supersaturated flowing stream of at least one solute in solution to oscillatory motion in an oscillating baffled reactor; applying ultrasonic radiation; and collecting the particles that are generated.

22. The process of claim 21, wherein the saturated or supersaturated flowing stream is formed by heating or cooling a stream of a solution of the solute.

23. The process of claim 21, wherein the saturated or supersaturated stream is formed by the process of claim 13.

24. A process for processing crystalline particles of at least one substance comprising subjecting a flowing stream comprising slurry of particles to oscillating motion in an oscillating baffled reactor; applying ultrasonic radiation and collecting the particles.

25. The process according to claim 24, wherein the size, surface morphology and/or crystallinity of the particles in the slurry are modified.

26. The process according to claim 24, wherein the particle slurry is produced by the process of claim 14.

27. Crystalline particles of at least one substance obtainable by the process of claim 14.

28. The crystalline particles suitable for inhalation according to claim 27 wherein the crystalline particles are 500 nm to 10 μm, preferably 600 nm to 5 μm, most preferably 650 nm to 2 μm.

29. The crystalline particles according to claim 27, wherein the crystalline particles are 2 μm to 900 μm, preferably 5 μm to 400 μm, most preferably 10 μm to 200 μm.

30. The crystalline particles according to claim 27, wherein the crystalline particles are selected from at least one active principal or desired precursor thereof.

31. The crystalline particles according to claim 27, wherein the crystalline particles are selected from the group consisting of an active pharmaceutical ingredient, an active agrochemical ingredient, a pharmaceutical excipient, an agrochemical excipient and appropriate mixtures of two or more thereof.

32. The crystalline particles according to claim 27, wherein the crystalline particles are a pharmaceutically active ingredient selected from the group consisting of anti-allergics, bronchodilators, anti-inflammatory steroids antibiotics, antivirals, antiinfectives, oncolytics, pain management medicines, Central Nervous System medicines, cardiovascular medicines, Parkinson disease medicines, HIV antivirals, epilepsy medicines, gastrointestinal medicines, musculoskeletal medicines, medicines for metabolic disorders, genitor-urinary medicines and orthopedic medicines and mixtures thereof.

33. The crystalline particles according to claim 27, wherein the crystalline particles are a pharmaceutically active ingredient suitable for use in an inhalation formulation, preferably fluticasone propionate, budesonide, salbutamol, formoterol or mixtures of two or more thereof.

34. The crystalline particles according to claim 27, wherein the crystalline particles are co-crystals containing an active principal or desired precursor thereof and at least one guest.

35. The crystalline particles according to claim 34, wherein the guest is an active principal or desired precursor thereof.
